(19) United States
(12) Patent Application Publication
(10) Pub. No.: US 2015/0328448 A1
(43) Pub. Date: Nov. 19, 2015

(54) ELECTRODE ELEMENT FOR ELECTROMEDICAL THERAPY IN A HUMAN OR ANIMAL BODY

(71) Applicant: BIOTRONIK AG, Buelach (CH)

(72) Inventors: Jan Helge Richter, Berlin (DE); Gernot Kolberg, Berlin (DE)

(21) Appl. No.: 14/690,629

(22) Filed: Apr. 20, 2015

Related U.S. Application Data
(60) Provisional application No. 61/992,248, filed on May 13, 2014.

(51) Int. Cl. A61N 1/05; A61N 1/375; A61N 1/365

(52) U.S. Cl. CPC A61N 1/056; A61N 1/36521; A61N 1/3756; A61N 1/0551

(57) ABSTRACT

An electrode element for electromedical therapy in a human or animal body, including an element body, on which at least one electric contact is arranged on an outer surface of the element body, wherein the at least one electric contact protrudes from the surface of the element body. The at least one electric contact has a raised contact body with an end face, on which at least one metal area is formed, wherein the metal area is surrounded by an electrical insulation. A catheter and a stimulation apparatus are also provided which include an electrode element.

### CROSS-REFERENCE TO RELATED APPLICATIONS

This patent application claims the benefit of co-pending U.S. Provisional Patent Application No. 61/992,248, filed on May 13, 2014, which is hereby incorporated by reference in its entirety.

### TECHNICAL FIELD

The present invention relates to an electrode element for electromedical therapy in a human or animal body, and to a catheter comprising an electrode element.

### BACKGROUND

A catheter comprising an electrode element for electromedical therapy with which raised electric contacts are arranged on the outer face of the catheter is known from U.S. Publication No. 2011/0301676. The electric contacts are placed annularly around the catheter and can be pressed against the wall of the respective vessel to be treated.

In the case of electromedical therapy, a current flow for stimulation, ablation and the like is applied by means of an electrode element introduced in situ.

However, parasitic currents, or what are known as shunt currents, which occur particularly frequently with laterally arranged electric contacts of this type and are caused by current flow via the blood in the area to be exposed to the therapy, are generally problematic. Shunt currents have a number of disadvantages. On the one hand, they make the applied therapeutic system inefficient because the shunt current is lost for the electromedical therapy. A pacemaker, for example, has to apply more energy than would be necessary for pure electromedical therapy. On the other hand, therapeutic success cannot be indicated directly from the energy balance during the therapy, since some of the applied energy is not used for the therapy. Further, shunt currents may lead to a coagulation of the blood.

The present invention is directed toward overcoming one or more of the above-mentioned problems.

### SUMMARY

An object of the present invention is to specify an improved electrode element and a catheter, an electrode or a device that can be implanted in a cable-free manner comprising an electrode element, with which shunt current problems are reduced, in particular, for heart electrodes, nerve electrodes, implantable sensors and leadless pacemakers.

At least the object is achieved in accordance with the present invention by the features of the independent claim(s). Favorable embodiments and advantages of the invention will emerge from the further claims, the description and the drawings.

An electrode element for electromedical therapy in a human or animal body is proposed, comprising an element body, on which at least one electric contact is arranged on an outer surface of the element body, wherein the at least one electric contact protrudes from the surface of the element body. The at least one electric contact has a raised contact body with an end face, on which at least one metal area is formed, wherein the metal area is surrounded by an electrical insulation.

As a result of the structure of the electrode element, the metal area can advantageously be placed against a natural wall of the body, for example, a vessel wall or the wall of an organ, such that the metal area is covered by the wall. The at least one metal area forms the actual electrode of the electrode element. Due to the electrical insulation, which is arranged around the at least one metal area, a current flow outside the contact region formed between the metal area and the wall is effectively reduced or even eliminated. Here, the insulation can be formed in one exemplary embodiment by an electrically insulating coating on the electric contact, or in another exemplary embodiment by an electrically insulating covering that is fastened to the electric contact, or in a further exemplary embodiment by an electrically insulating covering that is not in direct bodily contact with the electric contact, or also by combinations of at least two of the various exemplary embodiments.

Besides the energy saving by the avoidance of shunt currents, it is possible to identify more easily whether the electrode element is resting in the desired manner against the target object, for example, a vessel wall, an organ and the like, since, if the electrode element is resting against a wall, a significant change of impedance of the electrode element is to be recorded. The impedance or the impedance change can thus be interpreted by the user as a clear indicator for proof of the wall contact. This is true, in particular, for what is known as bipolar ablation, in which case a heart tissue, for example, is treated in order to interrupt the stimulus pathways thereof. Parallel ablation with a plurality of electrode pairs with use of just one generator is advantageous.

An electrically active area on the part of the electric contact in good mechanical contact with the tissue can advantageously be reduced. Furthermore, the electrode can be pressed against the surface of the tissue wall in an improved manner.

Favorable materials for the insulation in the metal area (electrode) are biocompatible materials. Favorable insulation materials include, for example, aluminum oxides, silicon carbide, diamond-like carbon (DLC), parylenes, silicones, polyurethanes, polysulfones, polycarbonates, polypropylenes, polymethylmethacrylates, PTFE (polytetrafluoroethylene), ETFE (ethylene tetrafluoroethylene), copolymers formed of silicones and polyurethanes, or also polyimide in suitable layer thicknesses Other insulation materials are contemplated. The insulation wall thicknesses of the edge insulation may advantageously be between 10 nm and 100 μm.

Favorable materials for the metal area include, for example, platinum, platinum iridium alloys, tungsten, tantalum, gold, palladium, steel, MP35N and Elgiloy.

In accordance with a favorable embodiment, at least edges around the metal area can be covered by the electrical insulation, at least in regions. An electrically active area on the part of the electric contact in good mechanical contact with the tissue can also favorably be reduced.

In accordance with a favorable embodiment, the electrical insulation of the at least one metal area of the electric contact can be formed at least in regions by the electrode body. An edge insulation on the electric contact can also optionally be combined with an insulation that is formed by the electrode body. In one embodiment, this may be the case on an individual contact in each case. In another embodiment, one or more electric contacts on the electrode body can be provided with edge insulation, and one or more other contacts can be insulated via the electrode body.

In accordance with a favorable embodiment, the contact body may be formed as a ring and may be placed around the element body. In particular, the electrical insulation may cover at least half of the ring in the peripheral direction, such that the metal area forms a ring segment.

With the embodiment of the electric contact body as a ring, which is electrically insulated radially on its surface to an extent greater than or equal to 180°, the remaining (electrically active) area is configured as a ring segment. Parallel leakage currents are thus avoided during therapy through the blood. The ring segment, similarly to the entire ring, is raised with respect to the element body or catheter shaft and can be positioned such that the electrically active area lies against a tissue wall after activation of the contact body or catheter shaft. Activation of the element body is understood to mean a structurally intended preferred form of the electrode element (for example, helix form), with which the contact body is pressed against the vessel wall. Since the impedance with complete wall contact rises, because any current has to flow through the tissue, which has a higher resistance compared to blood, it is possible on the one hand to check the wall contact by measuring the impedance before an energy application. On the other hand, the efficacy during therapy can be assessed by the determination of the introduced energy.

In accordance with a favorable embodiment, the electric contact can be arranged on an end face of the element body. In particular, the contact body can protrude from the end face of the element body. This embodiment is particularly suitable for leadless cardiac pacemakers (“leadless pacers”).

In accordance with a favorable embodiment, at least two electric contacts can be provided, which are arranged axially above one another on the element body, wherein two electric contacts may preferably have different polarities.

In accordance with a favorable embodiment, at least two electric contacts can be provided, which are axially and radially distanced from one another, wherein two electric contacts may preferably have different polarities. An electrode area that is shaped arbitrarily and that is simultaneously increased compared with the element body, which is shaft-shaped, for example, or compared with a catheter wall and that can be electrically insulated on the sides and is only electrically active at the area resting against the tissue, is largely insulated with respect to the blood.

In accordance with a favorable embodiment, an array of a plurality of electric contacts may be provided. An array or a series of a number of (at least two) raised metal areas (insulated toward the edge where applicable) of identical polarity has the advantageous effect that the relatively small areas can be brought into contact very easily with a wall of a large cohesive area (vessel wall, muscle, organ, etc.). The size of the metal areas determines the impedance of the system. With individual metal areas separate from one another, the impedance is much lower than would be expected for the sum of the impedances of the individual small metal areas. An electrode (metal area) that acts over a large area, but only has to bear against few points is created for the tissue. The shunt currents are thus significantly reduced.

In accordance with a favorable embodiment, the at least one electric contact can be spring-mounted. The spring-mounting can be produced by means of one or more spring elements, in, for example, an air-cushioned manner or liquid-assisted manner, such that the at least one electric contact can be pressed against the tissue reliably, even with unevennesses. By way of example, coil springs, leaf springs or flexible plastic can be provided as spring elements.

In accordance with a favorable embodiment, the at least one electric contact can be equipped with a surface structure, which at least doubles the effective contact area compared with macroscopic geometric dimensions. The enlarged surface can be produced by coating the metal area, for example, fractally. The enlarged surface advantageously means that an inevitably provided double-layer capacitance of the electrode element is considerably increased. In spite of a high charge shift, the polarization of the electrode area (metal area) is thus reduced very significantly. During the course of the electromedical therapy, high currents can thus flow without resulting in undesirable electrolytic reactions, which would irritate the tissue. In addition, the electrode areas can also be used, following an application of current, directly for the dissipation of low muscle potentials, without this being superimposed by polarization effects.

In accordance with a favorable embodiment, the contact body may have at least two separate metal areas, wherein the two separate metal areas are preferably provided for different polarities. In particular, the first of the areas may be substantially flat, and the second of the metal areas may protrude beyond the first metal area. The stimulating or ablating electrode may thus advantageously protrude three-dimensionally from the surface of the electrode body, whereas the second (counter) electrode lies in the plane of the wall.

A bipolar electric contact, which is produced in the form of a disk on the element body or the catheter shaft, in the center of which a pole protrudes in a raised manner from the disk, means that this pole can be pressed effectively into the tissue. Any current therefore necessarily flows via the tissue. The other pole can be located as a metal area at the surface of the element body or the catheter surface.

In accordance with a favorable embodiment, the contact body can be formed as a sleeve, wherein the metal area forms part of the lateral outer surface. Due to the “barrel-like” embodiment of the contact body, the electrically active lateral outer surface (metal area) thereof can protrude radially from the element body or catheter shaft and can be positioned such that the electrically active metal area lies against the tissue wall following activation of the element body or catheter shaft. Here, the sleeve can be formed with or without a flattened portion in the region of the protruding metal area.

In accordance with a further aspect of the present invention, a catheter comprising an electrode element is proposed. The electrode element can be connected to the catheter or integrated therein. Efficient electromedical therapy is possible, since parasitic shunt currents are effectively reduced or even eliminated as a result of the electrode element according to the invention. An electrode area that is shaped arbitrarily and that is simultaneously increased compared with the catheter wall and that can be electrically insulated on the sides and only electrically active at the area resting against the tissue is largely insulated with respect to the blood.

The electric contact can be formed as a “button”, with which the electrically active area is convex and is raised with respect to the element body or catheter shaft. The button is positioned such that the electrically active metal area lies against the tissue wall following activation of the element body or catheter shaft. The shape of the contact body and, in particular, of the metal area forming the electrode, can be selected in accordance with need, for example, can be rectangular, mushroom-shaped, can be formed as a rectangular disk, or as a round disk.

Further, a stimulation apparatus comprising an electrode element according to the present invention is proposed. The stimulation apparatus may be formed, in particular, as a leadless cardiac pacemaker or as a heart electrode.

In accordance with an advantageous method for operating an electrode element for electromedical therapy in a human or animal body with electrical energy, wherein the electrode element has at least one electric contact, an impedance of the at least one electric contact is used for energy regulation during the electric therapy. Electric energy is then advantageously only released in the suitable magnitude if at least the one electric contact has a minimum impedance. The impedance can be used in addition to or instead of the currently conventional parameters constituted by temperature and/or power for the regulation.

Further features, aspects, objects, advantages, and possible applications of the present invention will become apparent from a study of the exemplary embodiments and examples described below, in combination with the Figures, and the appended claims

### DESCRIPTION OF THE DRAWINGS

The present invention will be explained in greater detail hereinafter by way of example on the basis of exemplary embodiments illustrated in drawings, in which:

FIG. 1 schematically shows an isometric illustration of part of the catheter comprising an electrode element with annular electric contacts as electrodes.

FIG. 2 schematically shows an individual annular electric contact from FIG. 1 with metal area as contact area and electrical insulation.

FIG. 3 schematically shows an individual convex electric contact with end-face metal area and edge-side electrical insulation.

FIG. 4 schematically shows an isometric illustration of part of a catheter comprising an electrode element with the convex electric contacts as electrodes from FIG. 3, which are distanced from one another radially and axially on the surface of the electrode element.

FIG. 5 schematically shows an isometric illustration of part of a catheter comprising an electrode element with electric contacts, which are arranged axially above one another in pairs, wherein the pairs are distanced from one another radially.

FIG. 6 schematically shows an individual rectangular electric contact from FIG. 5 with end-face metal area and edge-side electrical insulation.

FIG. 7 schematically shows an isometric illustration of part of a catheter comprising an electrode element with rectangular electric contacts protruding from an area and arranged axially above one another, wherein the pairs are distanced from one another radially.

FIG. 8 schematically shows an individual rectangular electric contact from FIG. 7 with end-face metal area and edge-side electrical insulation.

FIG. 9 schematically shows an isometric illustration of part of a catheter comprising an electrode element with round, spherical electric contacts protruding from an area and arranged axially above one another, wherein the pairs are distanced radially from one another.

FIG. 10 schematically shows an individual electric contact from FIG. 9 with end-face metal area and edge-side electrical insulation over a metal area.

FIG. 11 schematically shows an isometric illustration of part of a catheter comprising an electrode element with round, button-shaped electric contacts, which are arranged axially above one another in pairs, wherein the pairs are distanced from one another radially.

FIG. 12 schematically shows an individual electric contact from FIG. 11 with end-face metal area and edge-side electrical insulation.

FIG. 13 schematically shows an isometric illustration of part of a catheter comprising an electrode element with sleeve-like electric contacts, which are arranged axially above one another, wherein the pairs are distanced from one another radially.

FIG. 14 schematically shows an individual electric contact from FIG. 13 with peripheral metal area and peripheral electrical insulation.

FIG. 15 schematically shows an alternative individual electric contact, for example for an arrangement as in FIG. 13, with flattened peripheral metal area and peripheral electrical insulation.

FIG. 16 schematically shows an isometric illustration of a stimulation apparatus comprising a bipolar electrode element, fitted at the end face, with two metal areas arranged side by side.

### DETAILED DESCRIPTION

In the Figures, functionally like or similarly acting elements are denoted in each case by like reference signs/numbers. The Figures are schematic illustrations of the present invention. They do not show specific parameters of the present invention, as these would be understood by one skilled in the art. Furthermore, the Figures merely show typical exemplary embodiments of the present invention and are not intended to limit the present invention to the illustrated embodiments.

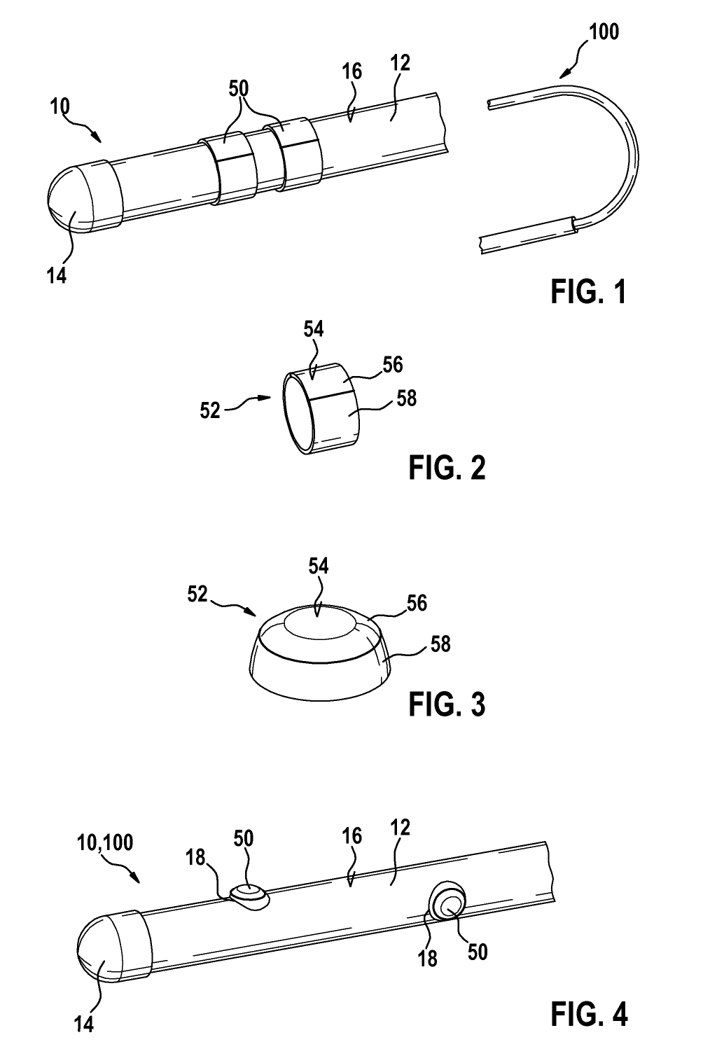

FIG. 1 shows an isometric illustration of part of a catheter 100 comprising an electrode element 10 for electric therapy in a human or animal body, with annular electric contacts 50 as electrodes. The electrode element 10 comprises an element body 12, which is terminated by a tip 14 and on which at least one electric contact 50 is arranged on an outer surface 16 of the element body 12, wherein the at least one electric contact 50 protrudes from the surface 16 of the element body 12. As shown in FIG. 2, the at least one electric contact 50 has a raised contact body 52 with an end face 54, on which a metal area 56 is formed, wherein the metal area 56 is surrounded by an electrical insulation 58.

FIG. 2, for illustration, shows an individual annular electric contact 50 from FIG. 1 with metal area 56 as contact area and with electrical insulation 58.

FIG. 3 shows an individual electric contact 50 with a convex contact body 52, which, on its end face 54, has a metal area 56, which is surrounded at the edge by an electrical insulation 58.

FIG. 4 shows an isometric illustration of part of a catheter 100 comprising an electrode element 10 for electric therapy in a human or animal body, with two convex electric contacts 50 from FIG. 3 as electrodes. The electrode element 10 comprises an element body 12, which is terminated by a tip 14 and on which the electric contacts 50 are arranged on an outer surface 16 of the element body 12. The electric contacts 50 protrude as electrodes from openings 18 from the surface 16 of the element body 12. The electric contacts 50 are distanced from one another radially and axially on the surface 16 of the electrode element 10.

In the further exemplary embodiments, the electrode elements 10 are structured identically apart from the electric contacts 50 and, therefore, only the differences in the exemplary embodiments will be discussed. All shown contact bodies 52 allow a reliable abutment of the electrode against the wall of an area to be treated, for example, a vessel wall, wherein parasitic shunt currents can be advantageously significantly reduced or eliminated.

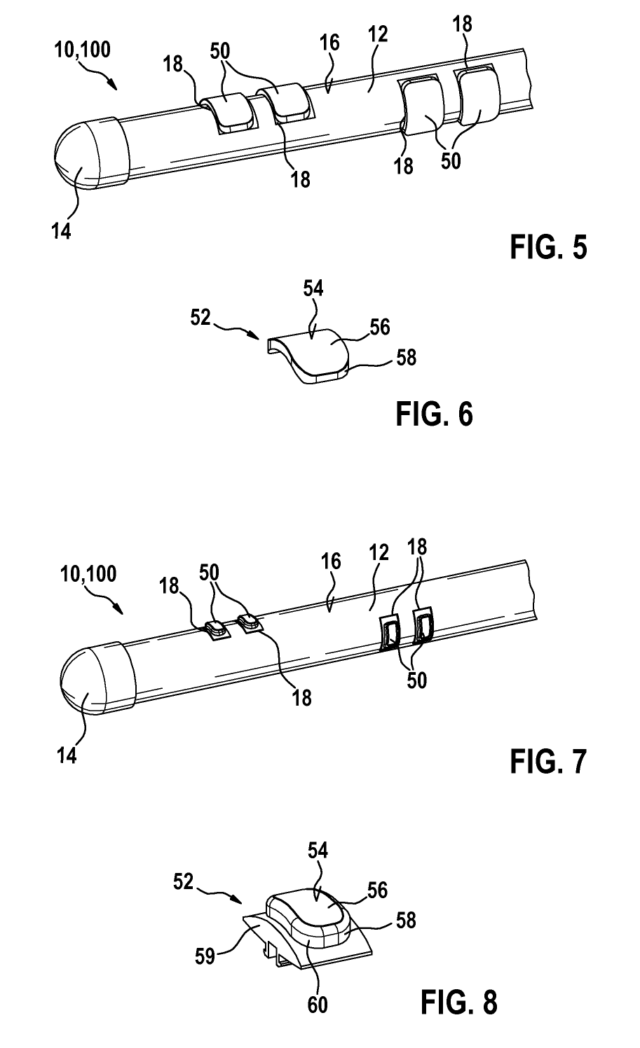

FIG. 5 shows an isometric illustration of part of a catheter 100 comprising an electrode element 10 with rectangular electric contacts 50 protruding from an area 59 (see FIG. 8). The electric contacts 50 are arranged axially above one another in pairs, wherein the pairs are distanced from one another radially. The electric contacts 50 in each of the pairs may have different polarity in each case.

FIG. 6 shows an individual rectangular electric contact 50 from FIG. 5 with metal area 56 on the end face 54 and edge-side electrical insulation 58. The contact body 52 is curved in accordance with a curvature of the element body 12.

FIG. 7 shows an isometric illustration of part of a catheter 100 comprising an electrode element 10 with rectangular electric contacts 50 protruding from an area 59 (see FIG. 8). The electric contacts 50 are arranged axially above one another in pairs, wherein the pairs are distanced from one another radially. The electric contacts 50 in each of the pairs may have different polarity in each case.

FIG. 8 shows an individual rectangular electric contact 50 from FIG. 7 with metal area 56 and edge-side electrical insulation 58. The contact body 52 has a base area 59, from which an elevation 60 protrudes, on the end face 54 of which the metal area 56 is arranged.

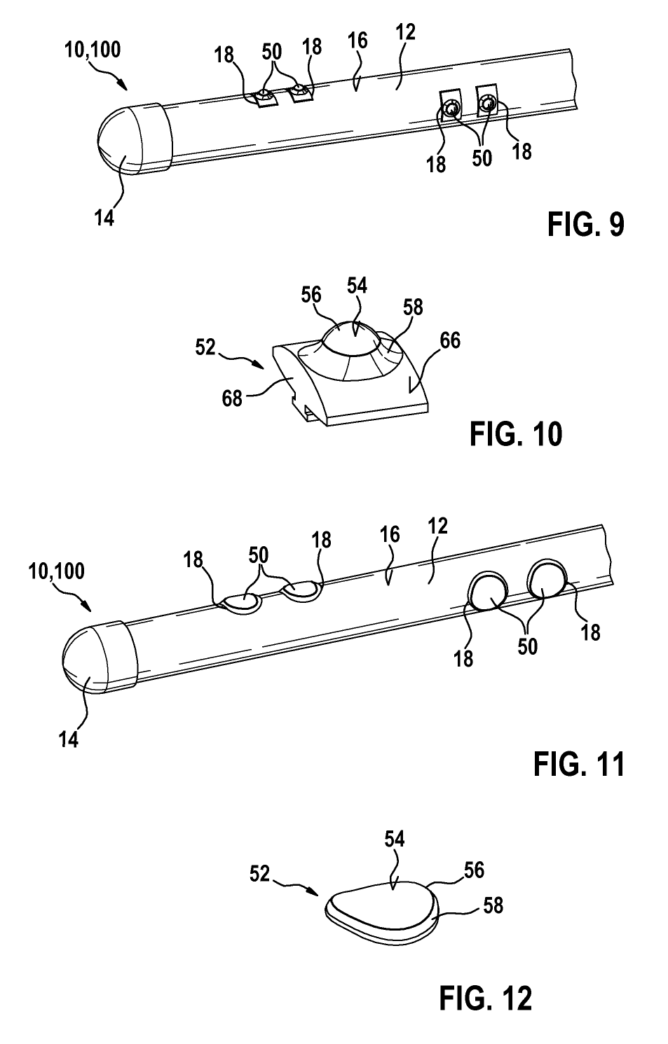

FIG. 9 shows an isometric illustration of part of a catheter 100 comprising an electrode element 10 with round, spherical electric contacts 50 protruding from an area. The electric contacts 50 are arranged axially above one another in pairs, wherein the pairs are distanced from one another radially. The individual electric contacts 50 in each of the pairs may have two polarities in each case.

FIG. 10 shows an individual electric contact 50 from FIG. 9 with end-face spherical metal area 56 and edge-side electrical insulation 58. The contact body 52 has a planar curved metal base area 66, beyond which the spherical metal area 56 rises. The electrical insulation 58 is formed between the area 56 and the base area 66. The base area 66 is likewise surrounded by an electrical insulation 68. Both metal areas 56, 66 form different electric poles, wherein the spherical area 56, for example, can be pressed directly into the myocardium during use and the base area 66 can be aligned with the surface 16 of the electrode element 10.

FIG. 11 shows an isometric illustration of part of a catheter 100 comprising an electrode element 10 with round, flat curved electric contacts 50. The electric contacts 50 are arranged axially above one another in pairs, wherein the pairs are distanced from one another radially. The electric contacts 50 in each of the pairs may have different polarity in each case.

FIG. 12 shows an individual electric contact from FIG. 11 with a metal area 56 on the end face 54 of the contact body 52, and an edge-side electrical insulation 58.

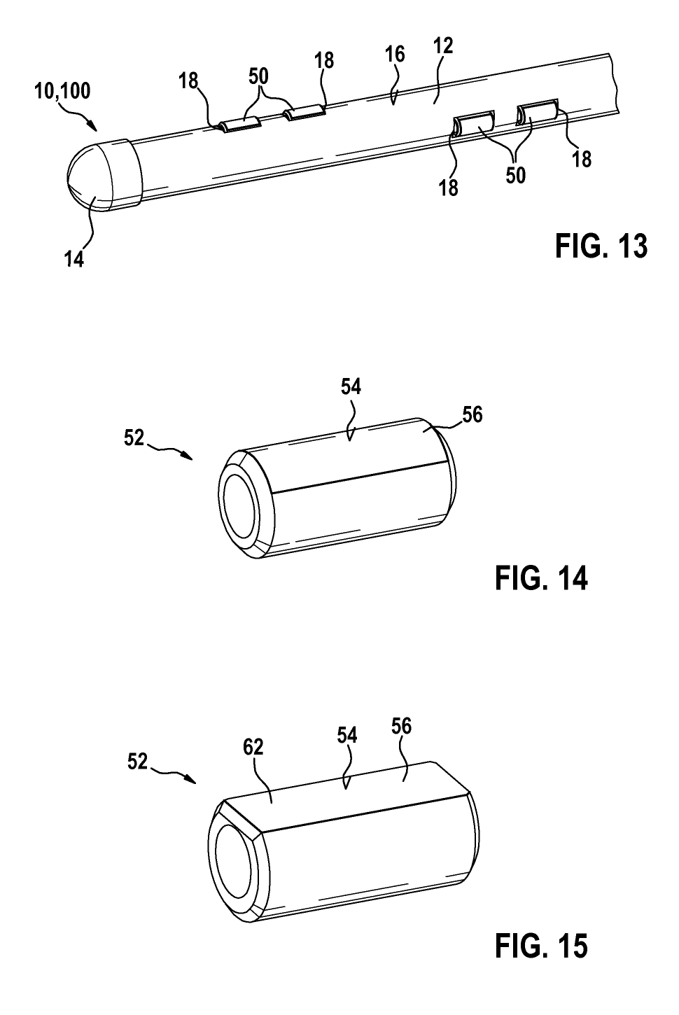

FIG. 13 shows an isometric illustration of part of a catheter 100 comprising an electrode element 10 with sleeve-shaped electric contacts 50, in which a lateral outer surface segment protrudes from an opening 18 in the surface 16 of the element body 12 in each case. The electric contacts 50 are arranged axially above one another in pairs, wherein the pairs are distanced from one another radially. The electric contacts 50 in each of the pairs may have different polarity in each case.

FIG. 14 shows an individual sleeve-shaped electric contact 50 from FIG. 13 with peripheral metal area 56 as an end face 54 on the contact body 52, such that the metal area 56 forms a lateral outer surface segment of the sleeve-shaped contact body 52. The metal area 56 extends, for example, over the entire axial length of the contact body 52. The rest of the surface of the contact body is covered by the electrical insulation 58.

FIG. 15 shows an alternative embodiment of a sleeve-shaped electric contact 50 of this type, in which the metal area 56 is arranged in a flattened region 62 of the contact body 52.

In addition, the metal areas 56, 66 described in the exemplary embodiments can be provided with a surface structure, which increases the surface of the metal areas compared with the macroscopic dimensions thereof.

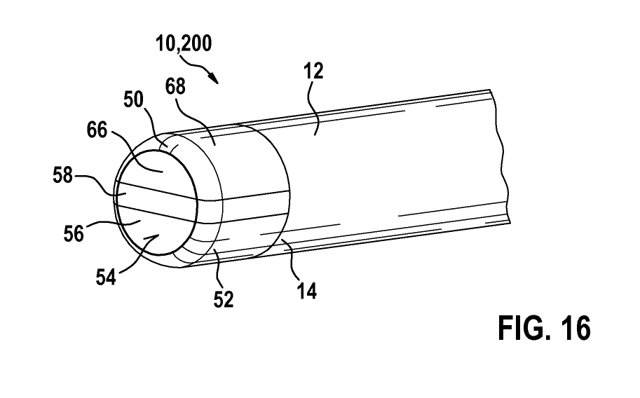

FIG. 16, by way of example, shows an isometric illustration of stimulation apparatus 200 comprising a bipolar electrode element 10 fitted on the end face and having two metal areas 56, 66 arranged side by side on the end face 54 of the contact body 52, the stimulation apparatus constituting a leadless cardiac pacemaker by way of example. Both metal areas 56, 66 form different electric poles. This electrode arrangement can also be applied in the case of heart electrodes. The contact body 52 of the electric contact 50 is arranged on the end face of the elongate element body 12. An electrical insulation 68 is provided on the side walls of the contact body 52. The two metal areas 56 arranged side by side are likewise electrically insulated from one another by an electrical insulation 58.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range.

### LIST OF REFERENCE NUMERALS

10 electrode element
12 element body
14 tip
16 surface
18 opening
50 electric contact
52 contact body
54 end face
56 metal area
58 electrical insulation
59 electrical insulation
69 area
60 collar
62 flattened region
66 metal area
68 electrical insulation
100 catheter comprising electrode element
200 leadless pacemaker

I/we claim:

1. An electrode element for electromedical therapy in a human or animal body, comprising:
an element body, on which at least one electric contact is arranged on an outer surface of the element body, wherein the at least one electric contact protrudes from the surface of the element body, wherein the at least one electric contact has a raised contact body with an end face, on which at least one metal area is formed, and wherein the metal area is surrounded by an electrical insulation.

2. The electrode element as claimed in claim 1, wherein at least edges around the metal area are covered by the electrical insulation, at least in regions.

3. The electrode element as claimed in claim 1, wherein the electrical insulation is formed at least in regions by the electrode body.

4. The electrode element as claimed in claim 1, wherein the contact body is formed as a ring and is placed around the element body.

5. The electrode element as claimed in claim 4, wherein the electrical insulation covers at least half of the ring in the peripheral direction, such that the metal area forms a ring segment.

6. The electrode element as claimed in claim 1, wherein at least two electric contacts are provided, which are arranged axially above one another on the element body, wherein two electric contacts have different polarities.

7. The electrode element as claimed in claim 1, wherein at least two electric contacts are provided, which are distanced from one another axially and/or radially, wherein two electric contacts have different polarities.

8. The electrode element as claimed in claim 1, wherein an array of a plurality of electric contacts is provided.

9. The electrode element as claimed in claim 1, wherein the at least one electric contact is spring-mounted.

10. The electrode element as claimed in claim 1, wherein the at least one electric contact is equipped with a surface structure that at least doubles the effective contact area compared with macroscopic geometric dimensions.

11. The electrode element as claimed in claim 1, wherein the contact body has at least two separate metal areas, wherein the two separate metal areas are provided for different polarities.

12. The electrode element as claimed in claim 11, wherein the first of the metal areas is substantially planar, and the second of the metal areas protrudes beyond the first metal area.

13. The electrode element as claimed in claim 1, wherein the contact body is formed as a sleeve, wherein the metal area forms part of the lateral outer surface of the sleeve.

14. A catheter comprising an electrode element as claimed in claim 1.

15. A stimulation apparatus, in particular a leadless pacemaker or heart electrode, comprising an electrode element as claimed in claim 1.

16. A method for operating an electrode element for electromedical therapy in a human or animal body with electrical energy, wherein the electrode element has at least one electric contact, wherein an impedance of the at least one electric contact is used for energy regulation during the electric therapy.

17. The method as claimed in claim 16, wherein electrical energy is then only released if the at least one electric contact has a minimum impedance.
